(19) United States
(12) Patent Application Publication
(10) Pub. No.: US 2010/0196510 A1
(43) Pub. Date: Aug. 5, 2010

(54) COMPOSITION AND TREATMENT

(75) Inventors: Ernst Wulfert, Brussels (BE); James Robert Murray, Bristol (GB)

Correspondence Address: KNOBBE MARTENS OLSON & BEAR LLP, 2040 MAIN STREET, FOURTEENTH FLOOR, IRVINE, CA, 92614, US

(73) Assignee: UMBERTO CORNELLI, MILANO (IT)

(21) Appl. No.: 12/670,633

(22) PCT Filed: Jul. 25, 2008
(86) PCT No.: PCT/GB08/50625

§ 371 Date: Mar. 12, 2010

(30) Foreign Application Priority Data
Jul. 25, 2007 (GB) 0714500.6

(51) Int. Cl. A61K 31/726; C08B 37/00; A61K 33/24; A61P 25/00

(52) U.S. Cl. 424/649; 536/55.1; 514/54

(57) ABSTRACT

Disclosed are compositions for the treatment of neuropathy and in particular peripheral neuropathy. The compounds include low molecular weight glycosaminoglycans. The peripheral neuropathy may be due to cancer treatment drugs.

The present disclosure generally relates to compositions for the treatment of neuropathy. The present disclosure relates to compounds such as low molecular weight glycosaminoglycans, compositions, kits and treatments for neuropathy and in particular peripheral neuropathy.

Neuropathy is any condition that affects any part of the nervous system and may be caused by, for example, disease, drug side effects or other external insult. The common causes of neuropathy are diabetes, herpes zoster infection, HIV-AIDS, toxins including neurotoxins, alcoholism, chronic trauma (such as repetitive motion disorders) or acute trauma (including surgery), and autoimmune conditions such as celiac disease, which can account for approximately 16% of small fiber neuropathy cases. Neuropathic pain is common in cancer as a direct result of the cancer on peripheral nerves (e.g., compression by a tumor), as a side effect of many chemotherapy drugs, and as a result of electrical injury. In peripheral neuropathy, patients can suffer from sensitivity losses in distal extremities followed by sensory ataxia. Axonal degeneration is also known to occur.

The disclosure relates generally to glycosaminoglycans, which are unbranched polysaccharides consisting of a repeating disaccharide unit. Heparin is one such glycosaminoglycan. The most common disaccharide unit in heparin is composed of a 2-O-sulfated iduronic acid and 6-O-sulfated, N-sulfated glucosamine, IdoA(2S)-GlcNS(6S). For example this makes up 85% of heparins from beef lung and about 75% of those from porcine intestinal mucosa. Other glycosaminoglycans include hyluronates, dermatan sulphates, chondroitin sulphates, heparan sulphates or keratan sulphates.

It is disclosed, in EP 0513513, that low molecular weight glycosaminoglycans having a molecular weight of 4500+/−1000 Daltons can be used in the treatment of diabetic neuropathy and diabetic nephropathy.

Even lower molecular weight glycosaminoglycans having an average molecular weight of 2400+/−200 Daltons, for the treatment of senile dementia and in particular, Alzheimer's disease, are disclosed in WO00/69444.

### STATEMENT OF DISCLOSURE

In one aspect, the disclosure relates to the use of glycosaminoglycans having a molecular weight of less than 3000 Daltons for the prevention or treatment of neuropathy; and to the use of glycosaminoglycans having a molecular weight of less than 3000 Daltons in the preparation of a medicament for the prevention or treatment of neuropathy.

In one aspect, the disclosure relates to the use of glycosaminoglycans having an average molecular weight of 2400 Daltons in the prevention or treatment of neuropathy; and to the use of glycosaminoglycans having an average molecular weight of 2400 Daltons in the preparation of a medicament for the prevention or treatment of neuropathy.

In one aspect, the disclosure relates to the use of glycosaminoglycans having no clinically significant anticoagulant activity or no anticoagulant activity in the prevention or treatment of neuropathy and to the use of glycosaminoglycans having no clinically significant anticoagulant activity or no anticoagulant activity in the preparation of a medicament for the prevention or treatment of neuropathy.

In one aspect the disclosure relates to a method for the prevention or treatment of neuropathy, the method comprising delivery to a subject in need thereof an effective amount of glycosaminoglycans of the present disclosure.

In one aspect the disclosure relates to a composition comprising glycosaminoglycans of the present disclosure in combination with a pharmaceutically acceptable diluent or excipient.

In one aspect the disclosure relates to a composition comprising glycosaminoglycans of the present disclosure in combination with a drug or agent that can cause neuropathy.

In one aspect the disclosure relates to a kit comprising glycosaminoglycans of the present disclosure and a drug or agent that can cause neuropathy.

In a further aspect, the neuropathy may be peripheral neuropathy. In another aspect, the peripheral neuropathy may be due to chemotherapy.

### DETAILED DESCRIPTION

The disclosure generally relates to the use of glycosaminoglycans, and in particular low molecular weight glycosaminoglycans, in the prevention or treatment of neuropathy such as peripheral neuropathy.

In one aspect the glycosaminoglycans have a molecular weight of less than 3000 Daltons, in one example more than 300 Daltons, in one example from 1500 to 3000 Daltons, in one example from 1900-2600 Daltons, in one example between 1920 Daltons to 2560 Daltons.

In one aspect the glycosaminoglycans have an average molecular weight of 2400 Daltons; in one example the average molecular weight is 2400±200 Daltons.

Molecular weight of the glycosaminoglycans may be determined by, for example, HPLC using columns for exclusion chromatography, as disclosed in WO00/69444, the disclosure of which is incorporated herein by reference.

In one aspect the glycosaminoglycans are obtained by depolymerization of one of the following starting materials: hyluronates, dermatan sulphates, chondroitin sulphates, heparin, heparan sulphates or keratan sulphates.

In one aspect the glycosaminoglycans are obtained from depolymerisation according to the methods disclosed in WO00/69444. The methods may be applied to all suitable starting materials.

In one aspect the glycosaminoglycans are prepared in part by an irradiation step. In one aspect the glycosaminoglycans are prepared in part by a gel permeation step or steps. In one aspect the glycosaminoglycans are prepared in part by an ultrafiltration step. A combination of steps may be used. In one example the glycosaminoglycan is obtained by depolymerization by treatment with radiation, suitably gamma radiation, optionally followed by a gel permeation step.

In one example the process for preparation of glycosaminoglycans comprises irradiation, then fractionation by gel permeation, ultrafiltration and further fractionation to produce glycosaminoglycans for use in the present disclosure.

In one aspect the starting material is heparin and the glycosaminoglycans are obtained from heparin depolymerisation as disclosed in WO00/69444.

Where heparin is used as a starting material it may be in the form of heparin sodium. In one aspect the heparin is from pig intestinal mucosa, for example having a molecular weight of 14000 Daltons and, for example, an activity of 190 u/mg. [One unit of heparin activity is generally defined as the quantity of heparin required to keep 1 ml of cats blood fluid for 24 hr at 0 degrees C. It is approximately equivalent approximately to 0.002 mg of pure heparin].

Any reference to depolymerisation of or purification of heparin to produce low molecular weight glycosaminoglycans, as given in more detail below, may also be taken to refer to depolymerisation of other suitable starting materials such as hyluronates, dermatan sulphates, chondroitin sulphates, heparin, heparan sulphates or keratan sulphates.

It will be understood by one skilled in the art that the glycosaminoglycans may be obtained by any method and from any suitable starting material and is not limited to the methods disclosed herein. The glycosaminoglycans may also be chemically synthesised by techniques known in the art.

One method for the preparation of glycosaminoglycans according to the disclosure is provided in WO00/69444. Other methods for obtaining low molecular weight glycosaminoglycans are described in, for example, EP0269937 and WO03/076474. The production method disclosed in WO00/69444 is described in detail below, for illustration only. The glycosaminoglycans of the present disclosure may have any or all of the features of the product of the method as disclosed in WO00/69444.

In one aspect of the present disclosure the glycosaminoglycans are made by or obtainable by the method of WO00/69444 and have all such features.

WO00/69444 specifically discloses that glycosaminoglycans having an average molecular weight of 2400 Daltons may be obtained by depolymerization of heparin according to a method including the following steps:

a) an aqueous solution of heparin is treated with gamma radiation from Co 60 according to the U.S. Pat. No. 4,987,222;
b) the solution obtained from the step a) is fractioned by gel permeation on Sephadex G/50 Medium resin;
c) the mixture of the fractions having molecular weight from 1,000 to 3,000 Daltons is ultrafiltered at 600 Daltons cut-off, washed and freeze-dried;
d) the freeze-dried product is dissolved and fractioned by gel permeation on Sephadex G/25 Medium resin;
e) the fractions having a molecular weight ranging from 1,920 to 2,560 Daltons, corresponding to an average molecular weight equal to 2,400 Daltons, are gathered and mixed.

For the preparation of the fraction of glycosaminoglycans having an average molecular weight equal to 2,400 Daltons, in a detailed embodiment, a 10% heparin aqueous solution is first treated with gamma radiation from Co 60 with an intensity ranging from 120 KGy to 150 KGy, at subsequent dosages of 25 KGy with respect to the heparin molecular weight.

The irradiated solution is ultrafiltered at 300 Daltons cut-off, purified, sterilely filtered and freeze-dried obtaining the “mother depolymerised” heparin.

This “mother depolymerised” heparin is dissolved in a 0.3 M solution of NaCl and then submitted to fractioning by gel permeation on Sephadex G/50 Medium resin.

The fractions having molecular weight<3,000 Daltons (about 10% of the total) form the raw material of the method.

The first treatment of this mixture consists in the ultrafiltration at 600 Daltons cut-off for the removal of the molecular fragments resulting from the depolymerization process.

The ultrafiltered mixture is washed with 0.3 M NaCl to the disappearance of the reaction to carbazole in the permeate.

The final mixture, taken to a concentration about equal to 8% in distilled water, is sterile filtered on 0.2 μm membranes and freeze-dried.

The freeze-dried product is dissolved in a 0.3 M NaCl solution at a concentration ranging from 10% to 15% (w/v).

In order to obtain a fraction having average molecular weight equal to 2,400 Daltons a second treatment of gel permeation is applied maintaining about the same parameters of the first gel permeation, using however the specific characteristics of the Sephadex G/25 Medium.

The used pilot column may be of the BP 252/15 kind with the following characteristics:

| | |
|---|---|
| Height | 105 cm |
| Resin volume | 5,200 mi |
| Flux | 1,000 ml/h |

The adopted parameters are represented by:

Ve=17,000 ml

Ve/Vo=1/R=1.26

R=0.79

K=0.09=(Ve−Vo):(Vt−Vo)

wherein:
Ve=elution volume
Vt=total volume of the resin
Vo=dead volume (initial solution in output)
R=solution (peak amplitude)

From the second gel permeation from 10 to 12 fractions are obtained comprising a series of molecular weights ranging from 3,000 Daltons to about 1,500 Daltons. For the preparation of the fraction of glycosaminoglycans only the fractions having molecular weights ranging from 1,920 Daltons to 2,560 Daltons are gathered and mixed.

The resulting solution is ultrafiltered by cut-off 300 to the removal of the sodium chloride.

The solution is submitted to concentration to about 10%, filtered at 0.2 μm and freeze-dried.

This fraction forms about 80% of the total of the fractions having molecular weight<3,000 Daltons.

WO00/69444 further characterises the product made using the above method.

In one aspect the glycosaminoglycans of the disclosure have one, or more, or all of the following physiochemical characteristics as disclosed in WO00/69444:

| | |
|---|---|
| Appearance | light yellow colour powder |
| Average molecular weight | 2,400 Daltons (+/−200) |
| Molecular weight distribution | 95% < 2,560 Daltons −> 1,920 Daltons |
| Polydispersion index | <1.20 |
| Organic sulphur | 9.5-11.5% |
| SO₃/COOH ratio | 2.3-2.6 |
| Specific rotation | >+35° |
| HPLC | see FIG. no. 1 of WO00/69444 |
| NMR | see FIG. no. 2 of WO00/69444 |

In WO00/69444 the average molecular weight was determined by HPLC using columns for exclusion chromatography, in comparison with LMW Heparins for Calibration Reference Substance Batch no. 1a (PH. EUR.) at molecular weight=3,700 Daltons.

In one aspect the glycosaminoglycans of the present disclosure have one, or more, or all of the following structural characteristics as disclosed in WO00/69444:

A profile having the characteristics of FIG. 1 of WO00/69444, which reports the plot obtained by HPLC of the fraction of glycosaminoglycans with the distribution of the molecular weights ranging for 95% from 2,560 Daltons to 1,920 Daltons, corresponding to about 4-5 constitutive disaccharides.

A profile having the characteristics of FIG. 2 of WO00/69444 which reports the NMR plot of the same fraction wherein one notices the absence of signals from 84 to 85 ppm characteristic of the linkage site which is removed by depolymerization with gamma radiation. The detachment of the galactosidic chain and its nitrogenous components represents a specific characteristic of this saccharidic fraction allowing to operate without the interferences, usually of pathological character, deriving from the presence of peptidic structures having different aminoacidic composition.

In WO00/69444 the NMR analysis reveals moreover that in the terminal zones there are only intact rings or eventually consisting of aliphatic “remnants”.

The rings of the reducing end are never desulfated.

Glucuronic structures desulfated at the reducing ends are not pointed out because they are destroyed by the gamma radiation.

In WO00/69444, in the rings of the not reducing ends, the terminal C4s are difficult to distinguish from the non-terminal C4s because they all fall into the same zone; the only ones which are identified in an unequivocal way are those ones of the NS, 3S glucosamine exactly adjacent to the glucuronic acid of the active pentamer. The structural cores correspond to the initial heparin with practically unaltered sulfation indexes. The anomeric signals deriving from the GlcNS0₃6SO₃ and IdoA2SO₃ reducing groups qualify the fraction of glycosaminoglycans as a substance with terminal units similar to those of the fragments from natural heparin.

In one aspect the glycosaminoglycans of the present disclosure have a polydispersion index lower than 1.20, in another aspect have a total absence of peptide components, in another aspect are free from desulfated units at the reducing end, obtained without resulfation treatment and in the absence of catalyzers.

In one aspect the glycosaminoglycans of the disclosure have one or more of the following biological characteristics disclosed in WO00/69444:

Ph. E. heparinic activity=absent
USP heparinic activity=absent
Anti Xa activity=<50 U aXa/mg
Anti IIa activity=<10 U alla/mg

Tests for heparinic activity (anti-coagulant activity) are well known in the art and to the skilled person. Any suitable test can be used.

For example, the prothrombin time (PT) test and the activated partial thromboplastin time (aPTT) test can be used to measure the global anticoagulant effects of heparin. The commercially available HEPTEST assay [American Diagnostica Inc] relies on the ability of heparin to catalyze the inactivation of exogenous bovine factor Xa by antithrombin III in the presence of naturally occurring plasma antagonist(s). The rate of factor Xa inhibition is directly proportional to the concentration of heparin present. This is indirectly measured by the prolongation of the recalcification time of the plasma sample. The HEPTEST assay consists of incubating an undiluted plasma or whole blood sample with an equal volume of factor Xa for a fixed time period at 37° C. This reaction mixture is then recalcified by the addition of RECALMIX a reagent containing optimal concentrations of calcium chloride and brain cephalin in a bovine plasma fraction rich in factor V and fibrinogen. The time it takes the plasma mixture to clot (in seconds) is then converted to units of heparin per ml plasma using a previously constructed standard calibration curve.

The anti-Xa assay is based on the ability of heparin to inhibit the activity of activated factor X (Xa) in a reagent. The reagent includes excess antithrombin, making the heparin in the sample the rate-limiting reagent for Xa inhibition.

In WO00/69444 it turns out that the fraction of glycosaminoglycans produced above proves the absence of a heparinic activity towards the parameters of the coagulation, notwithstanding the maintenance of the structural characteristics of the heparinic molecule.

In one aspect of the disclosure the glycosaminoglycans of the disclosure have no clinically significant anticoagulant activity, or no anticoagulant activity, for example as measured by the United States Pharmacopeia standard or other suitable validated assay.

In one aspect the glycosaminoglycans have a reduced anticoagulant activity in the same test, as compared to heparin, for example pig intestine heparin, or other suitable control.

In one aspect the glycosaminoglycans have Anti Xa activity=<50 U aXa/mg, and/or have Anti IIa activity=<10 U alla/mg.

In one aspect a clinically significant anticoagulant activity of glycosaminoglycans is considered to be activity that would prevent or teach away from use in a patient, for example because that anticoagulant effect would be of potential detriment or of no advantage to the patient.

Further aspects of the present disclosure include glycosaminoglycans with an average molecular weight of 2,400 Daltons with a polydispersion index lower than 1.20, and in one example an absence of peptidic components, and in a further example which are free from desulfated units at the reducing end.

In one aspect the disclosure relates to use of glycosaminoglycans as disclosed herein in the prevention or treatment of neuropathy.

In one aspect the neuropathy is peripheral neuropathy, which relates to the damage to nerves and neurons that reside or extend outside the central nervous system (the brain and spinal cord).

In one aspect the neuropathy is caused by, or may be caused by, an external agent, for example an agent not deriving from a tissue or organism, but which is administered or delivered or incident upon it and which can cause damage to nerves. The agent may be a drug or chemical which is delivered to a subject or tissue, for example.

The external agent may also be tissue trauma or the future risk of such trauma caused by, for example, surgery.

In one aspect the external agent is a chemotherapy agent. In one aspect the external agent is a DNA-crosslinking agent. In one aspect the external agent is an anti-mitotic compound. In one aspect the external agent is a DNA alkylating agent, for example a member of the “platin” group of compounds, such as cisplatin, [cisplatinum or cis-diamminedichloroplatinum(II) (CDDP)], nedaplatin, satraplatinum, carboplatin and oxaliplatin. The “platins” are non-cell cycle dependant alkylating agents. There is a direct interaction with DNA but cell death also occurs due to DNA binding and subsequent platinating of the DNA. At the molecular level, platinum compounds trigger the production of reactive oxygen and nitrogen species that induce membrane peroxidation overwhelming cellular defence mechanisms. Cisplatin is a prodrug where chlorine groups on the molecule are displaced within cells by highly reactive water ions. Only when the chlorine atoms are in the cis—form is it active (the isomer, transplatin, is not active). Cisplatin causes a variety of intrastrand and interstrand cross-links related to guanines or adjacent guanine/adenine components.

Glycosaminoglycans of the present disclosure may be used in treatment or prevention of neuropathy for any medical treatment or process which is known to be associated with neuropathy as a side effect, for example antiretroviral treatments such as Zerit, treatment with hydroxyurea plus didanosine and stavudine for HIV infection, dapsone, isoniazid, metronidazole and vincristine. Other treatments known to be associated with causing neuropathy, and in particular peripheral neuropathy are well known to the skilled person.

Glycosaminoglycans of the present disclosure can also be used to treat neuropathy resulting from herpes zoster infection, HIV-AIDS, toxins, alcoholism, chronic trauma (such as repetitive motion disorders) or acute trauma (including surgery), various neurotoxins and autoimmune conditions such as celiac disease.

In one aspect the external agent is an anticoagulant treatment, as compressive neuropathy can be a complication of anticoagulation.

In one aspect glycosaminoglycans used to treat or prevent neuropathy do not have a clinically significant anticoagulant effect, or have a reduced anticoagulant effect, or have no anticoagulant effect, when compared to heparin. This provides potential advantages when the cause of the neuropathy is an anticoagulant.

It will be appreciated by one skilled in the art that the low molecular weight glycosaminoglycans as disclosed herein can be used to treat or protect against peripheral neuropathy caused by any condition. The examples provided herein are by way of illustration only and the use of the glycosaminoglycans of the disclosure is not limited by the specific examples.

In one aspect the use of glycosaminoglycans in neuropathy according to the present disclosure contrasts with the neuropathy which is not induced by an external event, and which arises as a result of a disease such as diabetes, for example. The present disclosure allows predictable future neuropathic events to be prevented or ameliorated.

In particular the use of glycosaminoglycans of the present disclosure is contemplated where there is a risk of neuropathy occurring, even if neuropathy does not always subsequently occur. For example, certain drugs are associated with neuropathy as a side effect in certain cases, but this may not occur in all cases. Nevertheless glycosaminoglycans of the present disclosure may be used in a prophylactic sense, to reduce the risk of side effects.

Thus in one aspect, the disclosure relates to the use of glycosaminoglycans of the present disclosure in the reduction or prevention of drug-induced neuropathy and in one aspect relates to the prevention of neuronal damage caused by agents such as, cisplatin and antiretrovirals.

The present disclosure provides novel compositions as well as uses.

A further aspect of the disclosure relates to pharmaceutical compositions comprising glycosaminoglycans as disclosed herein, suitable for the treatment of neuropathy comprising an effective amount of a glycosaminoglycan in mixture with pharmaceutically acceptable diluents or excipients.

In a yet further aspect the disclosure relates to a composition comprising glycosaminoglycans of the present disclosure in combination with a drug or other agent that can cause neuropathy.

For the avoidance of doubt, glycosaminoglycans of the present disclosure are any of those disclosed herein for use in the treatment or prevention of neuropathy, including, for example, glycosaminoglycans having a molecular weight of less than 3000 Daltons; or glycosaminoglycans having an average molecular weight of 2400 Daltons; or glycosaminoglycans having an average molecular weight of 2400±200 Daltons; or glycosaminoglycans having no clinically significant anticoagulant activity or no anticoagulant activity; or in one example glycosaminoglycans having one or more of these properties.

By way of example we have demonstrated that the delivery of cisplatin in combination with glycosaminoglycans having an average molecular weight of 2400 Daltons can prevent neuropathic effects that are otherwise caused by cisplatin, as assessed by the methods disclosed in the specific examples.

In one aspect the combination comprises glycosaminoglycans of the present disclosure with any of the agents or drugs disclosed herein, or otherwise known to the skilled person, associated with neuropathy. Such agents or drugs may directly or indirectly cause neuropathy. The drug or other agent may be associated with neuropathy as a side effect of the drug treatment.

In one aspect the composition of the present disclosure comprises glycosaminoglycans of the present disclosure together with a chemotherapeutic agent associated with neuropathy. An example of such an agent is a member of the platin family, including cisplatin, nedaplatin, satraplatinum, carboplatin and oxaliplatin.

In one aspect the glycosaminoglycans are present in an amount suitable to prevent or ameliorate or treat the neuropathy associated with the agent or drug with which they are delivered.

In a further aspect the composition is a pharmaceutical composition which comprises a pharmaceutically acceptable excipient in combination with the glycosaminoglycans of the present disclosure and agent or drug.

In one aspect the disclosure further relates to a kit comprising glycosaminoglycans of the present disclosure and an agent, such as a drug, that causes neuropathy. The drug may be a platin such as, cisplatin, for example. In this case the drug and the glycosaminoglycans may be for substantially concomitant delivery or the glycosaminoglycans may be delivered before the delivery of the drug or agent which is associated with neuropathy.

The glycosaminoglycans of the present disclosure may be delivered by a different delivery vehicle when compared to the neuropathy causing agent. For example, the agent associated with neuropathy may be an oral agent, whereas the glycosaminoglycans may be delivered by injection, or other suitable route.

In one aspect the assessment of the prevention or treatment of neuropathy is based on the reduction of the loss of neurite density and/or neuronal cell death (loss of neuronal cell bodies). Suitable methods for assessment of these parameters are described herein, and include analysis of monoclonal anti MAP-2 antibody binding to cell bodies and anti β-tubulin antibody binding to neuritis. Analysis of the number of cell bodies labelled with anti MAP-2 antibodies, and total length of neurites labelled with anti β-tubulin antibodies can be carried out using, for example, the In Cell Analyzer 1000 3.2.Workstation software.

In one aspect the prevention or treatment of neuropathy using glycosaminoglycans can be assessed in cell culture by treating rat dorsal root ganglia with glycosaminoglycans as disclosed herein. Clinical trials can also be used to assess treatment of neuropathy, where appropriate.

Where the glycosaminoglycans of the disclosure are combined with an excipient, then suitable excipients for use in pharmaceutical compositions are well known and include saline, buffered saline, dextrose, water, glycerol, ethanol, and combinations thereof.

The pharmaceutical compositions may additionally comprise agents or drugs that are associated with neuropathy, as explained above.

The glycosaminoglycans of the disclosure or compositions comprising the glycosaminoglycans may be adapted to the route of administration, for instance the systemic or oral route. Forms of systemic administration include injection, typically intravenous injection. Other injection routes, such as subcutaneous, intramuscular, or intraperitoneal, can be used. Alternative means for systemic administration include transmucosal and transdermal administration. In addition, oral administration is also contemplated. Administration may also be topical and/or localized, in the form of salves, pastes, gels, solutions, powders and the like. The compositions of the disclosure may be delivered by a dosage form, including tablets, dispersions, suspensions, solutions, capsules, creams, ointments and aerosols.

For administration to mammals, and particularly humans, the daily dosage level of the active agent may be from 0.01 mg/kg to 10 mg/kg, typically around 1 mg/kg. The physician in any event will determine the actual dosage which will be most suitable for an individual and will vary with the age, weight and response of the particular individual. There can, of course, be individual instances where higher or lower dosage ranges are merited, and such are within the scope of this disclosure.

The dosage range required depends on the choice of the exact glycosaminoglycans, the route of administration, the nature of the formulation, the nature of the subject's condition, and the judgment of the attending practitioner.

A further aspect of the disclosure relates to a composition comprising an amount of said glycosaminoglycan equal to 10-200 mg per unit dose.

A further aspect of the disclosure relates to a therapeutic or prophylactic method for the treatment of patients suffering from or expected to suffer from neuropathy, consisting in the administration of an amount from 10 to 400 mg per day of a glycosaminoglycan of the present disclosure, optionally in the form of a pharmaceutical composition comprising a pharmaceutically acceptable diluents or excipients.

A further aspect of the disclosure relates to use of a glycosaminoglycan as disclosed herein in combination with another agent for the prevention or treatment of neuropathy, such as NGF, or serotonin-norepinephrine reuptake inhibitor (SNRI), such as Duloxetine. Other agents for the treatment of neuropathy are well known to the person skilled in the art.

It will be understood that the above description of aspects and embodiments is given by way of example only and that various modifications may be made by those skilled in the art. Individual features of certain embodiments may be combined with individual features of other embodiments, unless otherwise apparent from the disclosure. Where the term “comprising” is used, the disclosure also relates to embodiments which consist of or consist essentially of those elements disclosed.

The present disclosure is illustrated by reference to the following examples which are not limiting on the disclosure.

### EXAMPLES

In this study, neuroprotective effects of glycosaminoglycans having an average molecular weight of 2400 Daltons were evaluated on rat sensory neurons in a model of peripheral neuropathy comprising of cisplatin induce toxicity. The low molecular weight glycosaminoglycans are referred to in the examples as 2400 D.

Cisplatin is an antimitotic compound used for the treatment of cancers. However, its use is limited by several adverse-effects, among which are the peripheral neuropathies. Cisplatin-induced neuropathies are predominantly sensitive neuropathies. Patients suffer from sensitivity losses in distal extremities followed by sensory ataxia. Histological studies showed axonal degeneration (Thompson et al. (1)). In cell cultures, treatment of sensitive neurons by cisplatin induced a decrease of neurite network density followed by cell body degeneration (Gill et al. (2)). Nerve Growth Factor (NGF), a specific growth factor for sensory neurons, has protective effects on neurones against this intoxication. Sensory neurones intoxication in culture by cisplatin is thus an adequate model for the study of neuroprotective effects of compounds in peripheral neuropathies.

Primary cultures of dissociated dorsal root ganglia sensory neurones were incubated with 3 μg/ml cisplatin, with and without 2400 D for 48 hours and 72 hours and the following parameters were evaluated:

1. neurone cell bodies stained with an anti MAP 2 antibody (microtubule associated protein, and
2. neurite network density stained with an anti β-Tubulin antibody

### 1. Cultures of Neurones

Rat sensory neurones were prepared according to the method described by Hall et al 1997(3). Briefly, a female rat (15 days gestation) was killed by cervical dislocation (Rats Wistar; Janvier, Le Genest-St-Isle, France) and the foetuses removed from the uterus. Their spinal cords with the dorsal root ganglia (DRG) were removed and placed in ice-cold medium of Leibovitz (L15, Fisher 11415-049) containing Penicillin 50 Ul/ml—Streptomycin 50 μg/ml (PS, 1%) and bovine serum albumin (BSA 1%, Sigma A6003). The DRG were recovered and dissociated by trypsinisation for 20 min at 37° C. (trypsin EDTA 10×, 10%, Fisher 15400054) diluted in PBS without calcium and magnesium (Fisher 2007-03). The reaction was stopped by addition of Dulbecco modified Eagle medium (DMEM, Fisher 21969-035) containing DNase I grade II (0.1 mg/ml Roche diagnostic 104159) and foetal bovine serum (FBS 10%, Fisher 10270-098). The cell suspension was triturated with a 10 ml pipette and centrifuged at 350×g for 10 min at room temperature. The pellet of dissociated cells was then resuspended in defined culture medium.

Viable cells were counted in a Neubauer cytometer using the trypan blue exclusion test (Sigma) and seeded at a density of 30 000 cells/well in 96 well-plates (Nunc). Wells were pre-coated with poly-L-lysine (10 μg/ml, Sigma P2636) in ultra pure sterile water (Merck Eurolab 60759.01).

Cells were allowed to adhere for 2 h and maintained in a humidified incubator at 37° C. in 5% CO₂/95% air atmosphere.

2. Incubation of Neuronal Cultures with 2400 D

After 5 days of culture, the culture medium was changed into a defined culture medium following the different conditions described below:
Vehicle (DMSO 0.1%)
Vehicle (DMSO 0.1%)+cisplatin (3 μg/ml, Sigma ref: p4394)
Test compound 2400 D (100 nM, 10 nM and 1 nM)+cisplatin (3 μg/ml)
Reference compound NGF (10 ng/ml)+cisplatin (3 μg/ml)

Six wells per condition were carried out to assess neuronal survival. After 48 hours and 72 hours of incubation, the neuronal cells were fixed for 5 minutes in an ethanol/acetic acid solution (95%/5%) at −20° C. and rinsed 3 times in PBS.

For the control of the neurotrophic effect of compounds, NGF (10 ng/ml), and 2400 D (100 nM, 10 nM and 1 nM) were incubated during 48 hours and 72 hours. At the end of the incubation, the cells were fixed for 5 minutes in an ethanol/acetic acid solution (95%/5%) at −20° C. and rinsed 3 times in PBS.

### 3. Analysis of Number of Cell Bodies and Neurite Network Per Field

Cell bodies of sensory neurones were labelled by monoclonal anti MAP-2 antibody (Sigma M4403, see photograph 1A) and the neurite of sensory neurones were labelled by monoclonal β-Tubulin antibody (Sigma T8660). These antibodies were diluted at 1:400 in incubation solution (PBS with 5% of FCS and 0.1% of saponin, Sigma S-7900). These antibodies specifically label neurone cell bodies and neurites, respectively.

After 2 hours of incubation, the cells were washed in PBS and incubated with Alexa Fluor 488 goat anti mouse IgG (Molecular Probes A11001) diluted at 1:300 in incubation solution to reveal the MAP-2 and the β-Tubulin antibodies. Cell nuclei were stained with a fluorescent marker (Hoechst staining solution, SIGMA H6024, 1 μg/ml in incubation solution during 1 hour).

For each condition, 2 pictures per well (12 pictures per condition) were taken using In Cell Analyzer 1000 (Amersham Biosciences) controlled by the computer software In Cell Analyzer 1000 3.2. For the MAP-2 labelling, the magnification was ×10, for the β-Tubulin labelling, the magnification was ×20. For each labelling, all the images were taken in the same conditions.

Analysis of the number of cell bodies labelled with anti MAP-2 antibodies, and total length of neurites labelled with anti β-Tubulin antibodies, were carried out using In Cell Analyzer 1000 3.2.Workstation software. The results were expressed in percentage compared to the vehicle. The comparisons of each group were carried out using the unpaired T test.

### 4. Protection by 2400 D Against Cisplatin-Induced Loss of Neurite Density

i) Incubation with 3 μg/ml Cisplatin for 48 Hours—See Table 1

Neurite density expressed a mean of 5459 μm of total neurite length per field after incubation of sensory neurones with medium (“vehicle”), i.e. without cisplatin for 48 hours. Incubation with cisplatin for 48 hours reduced the total neurite length per field to approx. 4585 μm. This reduction in neurite network density by cisplatin was statistically significant (−16%, p<0.001) when compared to vehicle.

Incubation with 10 ng/ml NGF for 48 hours prevented cisplatin-induced loss of neurites and caused a significant increase in neurite length when compared with cultures incubated with medium only.

Incubation with 1 nM, and 10 nM 2400 D protected sensory neurones against cisplatin-induced neurite loss at 48 hours. This effect was statistically significant. Total neurite length was 5029 μm and 5071 μm after 48 hours of incubation with 1 nM and 10 nM 2400 D respectively, which represents a reduction of cisplatin-induced toxicity of respectively 49.3 and 44.5%.

ii) Incubation with 3 μg/ml Cisplatin for 72 Hours—See Table 2

Sensory neurones in medium “vehicle” without cisplatin expressed a mean of 5320 μm of total neurite length per field. Incubation with cisplatin for 72 hours reduced the total neurite length per field to approx. 4046 μm. This reduction in neurite network density by cisplatin was statistically significant (−24%, p<0.001) when compared to vehicle.

Incubation with 10 ng/ml NGF for 72 hours prevented cisplatin-induced loss of neurites and caused a significant increase in neurite length when compared with cultures incubated with medium only.

Incubation with 2400 D tested at 10 nM and 100 nM, protected sensory neurones against cisplatin-induced neurite loss at 72 hours. This effect was statistically significant. Total neurite length was 5125 μm and 4698 μm after 72 hours of incubation with 10 nM and 100 nM 2400 D respectively, which represents a reduction of cisplatin-induced toxicity of respectively 84.7 and 51.2%.

### 5. Protection by 2400 D Against Cisplatin-Induced Neuronal Cell Death (Loss of Neuronal Cell Bodies)

i) Incubation with 3 μg/ml Cisplatin for 48 Hours—See Table 3

A mean of 61 sensory neurones per field was observed after incubation in medium together with “vehicle” without cisplatin at 48 hours. Incubation with 3 μg/ml cisplatin, reduced the number of neurones to a mean of 40 sensory neurones per field was observed. This loss of neuronal cell bodies by cisplatin was statistically significant (−33%, p<0.001) when compared to the number of cell bodies assessed after 48 hours without cisplatin, i.e. medium containing vehicle only.

Incubation with 10 ng/ml NGF for 48 hours almost completely prevented cisplatin-induced loss of cell bodies.

Incubation with 1 nM, 10 nM and 100 nM 2400 D protected sensory neurones against cisplatin-induced cell death at 48 hours. This effect was statistically significant. Total number of sensory neurones per field was 49, 52 and 52 after 48 hours of incubation with 1 nM and 10 nM 2400 D respectively, which represents a reduction of cisplatin-induced cell death of 58.5%, 44% and 44% respectively.

ii) Incubation with 3 μg/ml Cisplatin for 72 Hours—See Table 4

A mean of 53 sensory neurones per field was observed after incubation in medium together with “vehicle” without cisplatin at 48 hours. Incubation with 3 μg/ml cisplatin, reduced the number of neurones to a mean of 32 sensory neurones per field. This loss of neuronal cell bodies by cisplatin was statistically significant (−39%, p<0.001) when compared to the number of cell bodies assessed after 48 hours without cisplatin, i.e. medium containing vehicle only.

Incubation with 10 ng/ml NGF for 48 hours completely prevented cisplatin-induced loss of cell bodies.

Incubation with 1 nM, 10 nM and 100 nM 2400 D completely protected sensory neurones against cisplatin-induced cell death at 72 hours. This effect was statistically significant. Total number of sensory neurones per field was 51, 52 and 54 after 72 hours of incubation with 1 nM, 10 nM and 100 nM 2400 D respectively, which represents a reduction of cisplatin-induced cell death of 90.5%, 95.2% and 104.7% respectively.

TABLE 1
Effects of the vehicle (0.1% DMSO), NGF (10 ng/ml) and 2400D (100
nM, 10 nM and 1 nM) on neurite length of sensory neurones after
48 hours of incubation in presence of cisplatin (3 μg/ml).

| | | | | sem | | | p |
|---|---|---|---|---|---|---|---|
| | | | | (standard | | % Control | (compared to |
| | | | Mean neurite | error | | without | vehicle + |
| Treatment | Conc. | | Length (μm) | mean) | n | cisplatin | cisplatin) |
| Vehicle | — | | 5459.72 | 184 | 12 | 100 | p < 0.001 |
| (0.1% DMSO) | | | | | | | |
| Vehicle | — | | 4585.52 | 133 | 12 | 84 | — |
| (0.1% DMSO) | | | | | | | |
| Intoxication with | | | | | | | |
| 3 μg/ml of cisplatin | | | | | | | |
| NGF | 10 | ng/ml | 6362.44 | 297 | 12 | 117 | p < 0.001 |
| Intoxication with | | | | | | | |
| 3 μg/ml of cisplatin | | | | | | | |
| 2400D | 100 | nM | 4761.13 | 142 | 12 | 87 | p > 0.05 |
| Intoxication with | 10 | nM | 5071.10 | 164 | 12 | 93 | p < 0.05 |
| 3 μg/ml of cisplatin | 1 | nM | 5029.10 | 85 | 12 | 92 | p < 0.05 |

TABLE 2
Effects of the vehicle (0.1% DMSO), NGF (10 ng/ml) and 2400D (100
nM, 10 nM and 1 nM) on the neurite length of sensory neurones after
72 hours of incubation in presence of cisplatin (3 μg/ml).

| | | | | sem | | | p |
|---|---|---|---|---|---|---|---|
| | | | | (standard | | % Control | (compared to |
| | | | Mean neurite | error | | without | vehicle + |
| Treatment | Conc. | | Length (μm) | mean) | n | cisplatin | cisplatin) |
| Vehicle | — | | 5320.43 | 156.74 | 12 | 100 | p < 0.001 |
| (0.1% DMSO) | | | | | | | |
| Vehicle | — | | 4046.14 | 138.52 | 12 | 76 | — |
| (0.1% DMSO) | | | | | | | |
| Intoxication with | | | | | | | |
| 3 μg/ml of cisplatin | | | | | | | |
| NGF | 10 | ng/ml | 6000.30 | 221.31 | 12 | 113 | p < 0.001 |
| Intoxication with | | | | | | | |
| 3 μg/ml of cisplatin | | | | | | | |
| 2400D | 100 | nM | 4698.37 | 94.79 | 12 | 88 | p < 0.001 |
| Intoxication with | 10 | nM | 5125.55 | 129.07 | 12 | 96 | p < 0.001 |
| 3 μg/ml of cisplatin | 1 | nM | 4356.65 | 139.01 | 12 | 82 | p > 0.05 |

TABLE 3
Effects of the vehicle (0.1% DMSO), NGF (10 ng/ml) and 2400D
(100 nM, 10 nM and 1 nM) on the number of cell bodies per field
after 48 hours of incubation with cisplatin (3 μg/ml).

| | | | | sem | | | p |
|---|---|---|---|---|---|---|---|
| | | | | (standard | | % Control | (compared to |
| | | | | error | | without | vehicle + |
| Treatment | Conc. | | Average | mean) | n | cisplatin | cisplatin) |
| Vehicle | — | | 60.83 | 1.59 | 12 | 100 | p < 0.001 |
| (0.1% DMSO) | | | | | | | |
| Vehicle | — | | 40.75 | 1.88 | 12 | 67 | — |
| (0.1% DMSO) | | | | | | | |
| Intoxication with | | | | | | | |
| 3 μg/ml of cisplatin | | | | | | | |
| NGF | 10 | ng/ml | 58.33 | 1.38 | 12 | 96 | p < 0.001 |
| Intoxication with | | | | | | | |
| 3 μg/ml of cisplatin | | | | | | | |
| 2400D | 100 | nM | 51.58 | 0.53 | 12 | 85 | p < 0.001 |
| Intoxication with | 10 | nM | 52.00 | 1.51 | 12 | 85 | p < 0.001 |
| 3 μg/ml of cisplatin | 1 | nM | 49.08 | 2.27 | 12 | 81 | p < 0.01 |

TABLE 4
Effects of the vehicle (0.1% DMSO), NGF (10 ng/ml) and 2400D
(100 nM, 10 nM and 1 nM) on the number of cell bodies per field
after 72 hours of incubation with cisplatin (3 μg/ml).

| | | | | sem | | | p |
|---|---|---|---|---|---|---|---|
| | | | | (standard | | % Control | (compared to |
| | | | | error | | without | vehicle + |
| Treatment | Conc. | | Average | mean) | n | cisplatin | cisplatin) |
| Vehicle | — | | 53.75 | 1.29 | 12 | 100 | p < 0.001 |
| (0.1% DMSO) | | | | | | | |
| Vehicle | — | | 32.83 | 1.47 | 12 | 61 | — |
| (0.1% DMSO) | | | | | | | |
| Intoxication with | | | | | | | |
| 3 μg/ml of cisplatin | | | | | | | |
| NGF | 10 | ng/ml | 54.92 | 1.41 | 12 | 102 | p < 0.001 |
| Intoxication with | | | | | | | |
| 3 μg/ml of cisplatin | | | | | | | |
| 2400D | 100 | nM | 54.67 | 1.60 | 12 | 102 | p < 0.001 |
| Intoxication with | 10 | nM | 52.58 | 1.62 | 12 | 98 | p < 0.001 |
| 3 μg/ml of cisplatin | 1 | nM | 51.25 | 1.47 | 12 | 95 | p < 0.001 |

Whilst the examples use 2400 D, which comprises low molecular weight glycosaminoglycans having an average molecular weight of 2400 Daltons±200 Daltons, it will be appreciated that other suitable glycosaminoglycans may also be used including those generally disclosed herein.

### REFERENCES

1 Thompson S W, Davis L E, Kornfeld M, Hilgers R D, Standefer J C Cisplatine neuropathy. Clinical, electrophysiologic, morphologic, and toxicological studies Cancer. 1984 Oct. 1; 54(7):1269-75.
2 Gill J S, Windebank A J. Cisplatine-induced apoptosis in rat dorsal root ganglion neurones is associated with attempted entry into the cell cycle J Clin Invest. 1998 Jun. 15; 101(12):2842-50.
3 Hall A K, Ai X, Hickman G E, MacPhedran S E, Nduaguba C O, Robertson C P. The generation of neuronal heterogeneity in a rat sensory ganglion. J Neurosci. 1997 Apr. 15; 17(8):2775-84.

1. Glycosaminoglycans having a molecular weight of less than 3000 Daltons.

2-15. (canceled)

16. pharmaceutical composition comprising glycosaminoglycans as defined in claim 1 in combination with a pharmaceutically acceptable diluent or excipient.

17. A pharmaceutical composition comprising glycosaminoglycans as defined in claim 1 in combination with a drug or other agent that can cause neuropathy.

18. A pharmaceutical composition according to claim 17 additionally comprising a pharmaceutically acceptable diluent or excipient.

19. A kit comprising glycosaminoglycans as defined in claim 1 and a drug or other agent that can cause neuropathy.

20. The pharmaceutical composition according to claim 17 wherein the drug is a platin family member.

21. The pharmaceutical composition according to claim 20 wherein the drug is selected from cisplatin, nedaplatin, satraplatinum, carboplatin or oxaliplatin.

22. (canceled)

23. The kit according to claim 19, wherein the drug is a platin family member.

24. The kit according to claim 23, wherein the drug is selected from cisplatin, nedaplatin, satraplatinum, carboplatin or oxaliplatin.

25. A method for the prevention or treatment of neuropathy, the method comprising the delivery to a subject in need thereof of an effective amount of glycosaminoglycans according to claim 1.

26. The method according to claim 25 wherein the glycosaminoglycans have an average molecular weight of about 2400 Daltons.

27. The method according to claim 26 wherein the glycosaminoglycans have an average molecular weight of 2400±200 Daltons.

28. The method according to claim 25 wherein the glycosaminoglycans have no clinically relevant anticoagulant activity or no anticoagulant activity.

29. The method according to claim 25, wherein the glycosaminoglycans are obtained by depolymerization of heparin.

30. The method according to claim 25, wherein the glycosaminoglycans have a polydispersion index of lower than 1.20, an absence of peptide components and are free from desulfated units at the reducing end.

31. The method according to claim 25, wherein the neuropathy is peripheral neuropathy.

32. The method according to claim 25, wherein the neuropathy is caused by the administration of, or exposure to, an external agent.

33. The method according to claim 32, wherein said external agent is selected from a chemotherapeutic agent, a DNA-crosslinking agent, an anti-mitotic compound, and a DNA alkylating agent.

34. The method according to claim 32, wherein said external agent is a drug of the platin family selected from cisplatin, nedaplatin, satraplatinum, carboplatin and oxaliplatin.

35. The method according to claim 31, wherein the glycosaminoglycans as defined in claim 1 are administered before delivery of said external agent.

36. The method according to claim 33, wherein the glycosaminoglycans as defined in claim 1 are administered in admixture with or concomitantly with said external.

37. A method for the prevention or treatment of neuropathy, the method comprising the delivery to a subject in need thereof of a pharmaceutical composition comprising glycosaminoglycans as defined in claim 1 and a pharmaceutically acceptable diluent or excipient.

38. The method according to claim 25, wherein the glycosaminoglycans are delivered in combination with another agent known for the prevention or treatment of neuropathy.
